(19) United States
(12) Patent Application Publication
(10) Pub. No.: US 2024/0127937 A1
(43) Pub. Date: Apr. 18, 2024

(54) DIGITAL MEDICAL IMAGE TEACHING AND RESEARCH SYSTEM

(71) Applicant: UNION HOSPITAL TONGJI MEDICAL COLLEGE HUST, Wuhan (CN)

(72) Inventors: Juan TAO, Wuhan (CN); Jinjin ZHU, Wuhan (CN); Liu YANG, Wuhan (CN); Jing YANG, Wuhan (CN); Yan LI, Wuhan (CN)

(73) Assignee: UNION HOSPITAL TONGJI MEDICAL COLLEGE HUST, Wuhan (CN)

(21) Appl. No.: 18/332,798

(22) Filed: Jun. 12, 2023

(30) Foreign Application Priority Data
Oct. 17, 2022 (CN) 202211268259.5

(51) Int. Cl. G16H 40/20; G16H 30/20

(52) U.S. Cl. CPC G16H 40/20; G16H 30/20

(57) ABSTRACT

Some embodiments of the disclosure provide a digital medical image teaching and research system. In some examples, the system includes a management side, a learning side, and a central processing unit. Both the management side and the learning side are connected to the central processing unit. In other examples, the management side includes an administrator login module, a management side control module, a teaching video module, a database, and a network connection module. The management side is configured to acquire medical image data and to generate a teaching video for scientific research personnel to perform scientific research and learning personnel to learn. The learning side includes a learning login module, a teaching video application module, and a learning side control module. The learning side is used for learning the teaching video. In further examples, a scientific research side is for the scientific research personnel to perform scientific research.

### CROSS REFERENCE TO RELATED APPLICATIONS

This application claims priority to Chinese patent application number 202211268259.5, filed on Oct. 17, 2022, the disclosure of which is incorporated by reference herein in its entirety.

### FIELD OF THE DISCLOSURE

The disclosure relates generally to the field of medical image teaching and research. More specifically, the disclosure relates to digital medical image teaching and research systems.

### BACKGROUND

Medicine is a discipline with a strong combination of practice and theory. Pure theoretical teaching cannot meet the needs of medical teaching, and pure theoretical research cannot meet the needs of medical scientific research. The existing medical experiment courses can carry out only basic operation simulation, but cannot implement the experimental courses for diagnosis and treatment effects. In addition, medical personnel often employ digital medical image software for learning. However, the existing digital medical image software is highly specialized. When in use, medical personnel need to be trained with diverse content of a plurality of digital medical image professional software, which is time-consuming and has low learning efficiency. Therefore, it is necessary to design a digital medical image teaching and research system.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a digital medical image teaching and research system, including a management side, a learning side, and a central processing unit, where both the management side and the learning side are connected to the central processing unit.

In other embodiments, the management side includes an administrator login module, a management side control module, a teaching video module, a database, and a network connection module, where the administrator login module, the teaching video module, the database, and the central processing unit are connected to the management side control module, the management side control module is connected to a hospital picture archiving and communication system (PACS) database through the network connection module, the administrator login module is for an administrator to manage account registration and login, the network connection module is configured to acquire medical image data in the hospital PACS database, the database is configured to store the medical image data acquired by the network connection module, and the teaching video module is configured to generate a teaching video according to the medical image data.

In further embodiments, the learning side includes a learning login module, a teaching video application module, and a learning side control module, where the learning login module, the teaching video application module, and the central processing unit are connected to the learning side control module, the learning login module is for learning personnel to register and login a learning account, and the teaching video application module is configured to acquire and play the teaching video.

Optionally, the network connection module includes a data acquisition front-end processor, a one-way isolation gatekeeper, a data sending front-end processor, and a firewall security device, where the hospital PACS database is connected to the data acquisition front-end processor through a PACS database interface, the data acquisition front-end processor is connected to the one-way isolation gatekeeper, the one-way isolation gatekeeper is connected to the data sending front-end processor, the data sending front-end processor is connected to the firewall security device, the firewall security device is connected to the administrator control module, the data acquisition front-end processor is configured to acquire the medical image data imported from the PACS database interface and transmit the medical image data to the data sending front-end processor through the one-way isolation gatekeeper, the one-way isolation gatekeeper is configured to ensure that the medical image data is not returned to the PACS database interface, the data sending front-end processor is configured to store the acquired medical image data into the database via the administrator control module, and the firewall security device is configured to ensure security of the database.

Optionally, the teaching video module includes a teaching video establishment module and a teaching video classification module, where the teaching video establishment module and the teaching video classification module are connected to the management side control module, the teaching video establishment module is configured to generate the teaching video according to the medical image data, and the teaching video classification module is configured to classify the teaching video according to types of the teaching video.

Optionally, the teaching video application module includes a pulmonary digital medical image module, an oral digital medical image module, a cardiac digital medical image module, a brain digital medical image module, an abdominal digital medical image module, an orthopedic digital medical image module, a gynecological digital medical image module, a three-dimensional (3D) printing modeling module, and a virtual-reality (VR) surgical image training module, where the pulmonary digital medical image module is configured to display a pulmonary medical image teaching video, the oral digital medical image module is configured to display an oral medical image teaching video, the cardiac digital medical image module is configured to display a cardiac digital medical image teaching video, the brain digital medical image module is configured to display a brain digital medical image teaching video, the abdominal digital medical image module is configured to display an abdominal medical image teaching video, the orthopedic digital medical image module is configured to display an orthopedic digital medical image teaching video, the gynecological digital medical image module is configured to display a gynecological digital medical image teaching video, the 3D printing modeling module is configured to implement 3D printing modeling, and the VR surgical image training module is configured to display a VR surgical image training teaching video.

Optionally, the digital medical image teaching and research system further includes a scientific research side, where the scientific research side includes a scientific research personnel login module, a scientific research control module, a scientific research log module, and a scientific research module, where the scientific research personnel login module, the scientific research log module, the scientific research module, and the central processing unit are connected to the scientific research control module, the scientific research personnel login module is for scientific research personnel to register and log in, the scientific research module is for the scientific research personnel to acquire and perform scientific research on the medical image data, and the scientific research log module is configured to view a scientific research operation record on the medical image data.

In some embodiments, the digital medical image teaching and research system provided by the present disclosure includes a management side, a learning side, a scientific research side, and a central processing unit. The management side is configured to acquire medical image data and generate a teaching video for scientific research personnel to perform scientific research and learning personnel to learn. The management side includes an administrator login module, a management side control module, a teaching video module, a database, and a network connection module. The administrator login module is for an administrator to manage account registration and login. The network connection module is configured to acquire the medical image data in a hospital PACS database. The database is configured to store the medical image data. The teaching video module is configured to generate the teaching video. The learning side is used for learning of the teaching video. The learning side includes a learning login module, a teaching video application module, and a learning side control module. The learning login module is configured to register and login a learning account. The teaching video application module is configured to acquire and play the teaching video. The scientific research side is for the scientific research personnel to perform scientific research. The scientific research side includes a scientific research personnel login module, a scientific research control module, a scientific research log module, and a scientific research module. The scientific research personnel login module is for the scientific research personnel to register and log in. The scientific research module is configured to acquire and perform scientific research on the medical image data. The scientific research log module is configured to view a scientific research operation record on the medical image data. The system may implement teaching and research based on medical images, may be easy to use, and may improve the efficiency of learning and scientific research.

### BRIEF DESCRIPTION OF THE DRAWINGS

Illustrative embodiments of the present disclosure are described in detail below with reference to the attached drawing figures.

FIG. 1 is a schematic structural diagram of a digital medical image teaching and research system according to an embodiment of the present disclosure.

FIG. 2 is a schematic structural diagram of a management side according to an embodiment of the present disclosure.

FIG. 3 is a schematic structural diagram of a learning side according to an embodiment of the present disclosure.

FIG. 4 is a schematic structural diagram of a scientific research side according to an embodiment of the present disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

The technical solutions of the embodiments of the present disclosure are described below with reference to the drawings. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

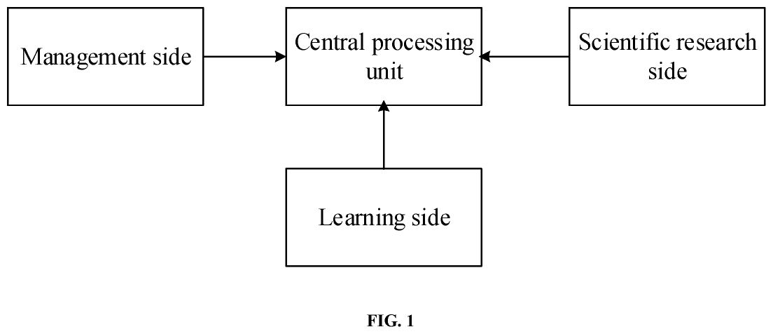

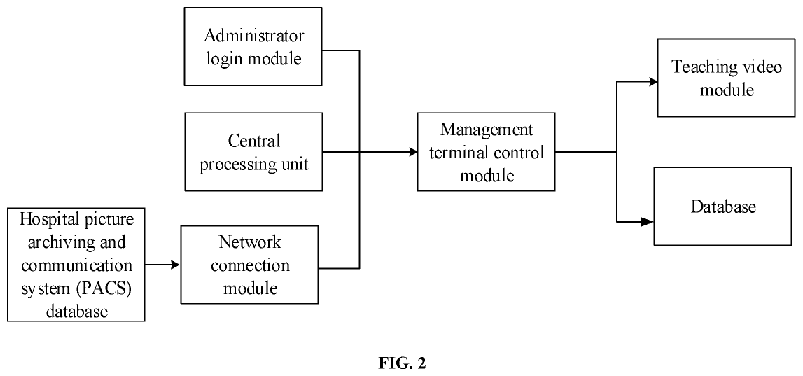

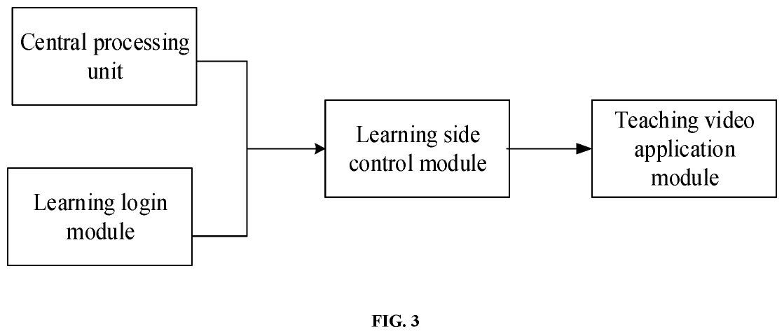

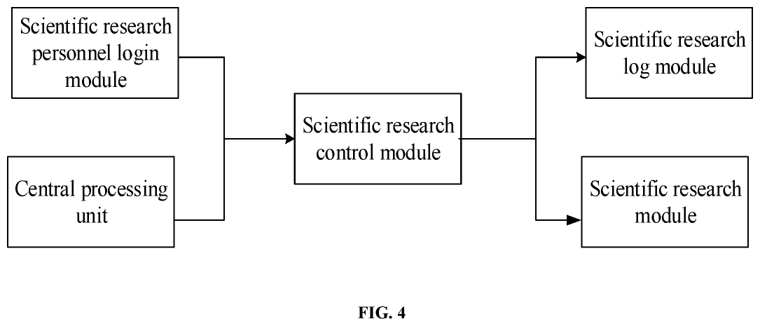

As shown in FIG. 1 to FIG. 4, the digital medical image teaching and research system provided in the embodiments of the present disclosure may include a management side, a learning side, and a central processing unit. Both the management side and the learning side are connected to the central processing unit.

The management side may include an administrator login module, a management side control module, a teaching video module, a database, and a network connection module, where the administrator login module, the teaching video module, the database, and the central processing unit are connected to the management side control module, the management side control module is connected to a hospital picture archiving and communication system (PACS) database through the network connection module, the administrator login module is for an administrator to manage account registration and login, the network connection module is configured to acquire medical image data in the hospital PACS database, the database is configured to store the medical image data acquired by the network connection module, and the teaching video module is configured to generate a teaching video according to the medical image data.

The specific process is as follows: the management side control module acquires the medical image data in the hospital PACS database through the network connection module and then stores same into the database, the management side control module acquires the medical image data in the database and sends same to the teaching video module, and the teaching video module generates the teaching video according to the medical image data. The teaching video may be generated by a teacher explaining the medical image data; or, a medical image data recognition model may be constructed through a depth learning algorithm, and the medical image data is recognized through the model to generate the teaching video. Herein, both are the prior art and are therefore not described in detail. After the teaching video is generated, the teaching video module classifies the medical image data according to its type, for example, classifying into an abdomen, a brain, an oral cavity, etc. according to organs. The management side control module acquires the teaching video and stores same into the database for saving.

The learning side may include a learning login module, a teaching video application module, and a learning side control module. The learning login module, the teaching video application module, and the central processing unit are connected to the learning side control module, the learning login module is for learning personnel to register and login a learning account, and the teaching video application module is configured to acquire and play the teaching video.

The specific process is as follows: the learning personnel select the needed type of teaching video according to specific requirements after registering and logging in. The learning side control module sends an instruction to the central processing unit, the central processing unit sends the instruction to the management side control module, the management side control module acquires the required teaching video from the database and sends same to the learning side control module through the central processing unit, and the learning side control module sends the teaching video to a different teaching video application module according to its type for the learning personnel to learn.

The network connection module may include a data acquisition front-end processor, a one-way isolation gatekeeper, a data sending front-end processor, and a firewall security device. The hospital PACS database is connected to the data acquisition front-end processor through a PACS database interface, the data acquisition front-end processor is connected to the one-way isolation gatekeeper, the one-way isolation gatekeeper is connected to the data sending front-end processor, the data sending front-end processor is connected to the firewall security device, the firewall security device is connected to the administrator control module, the data acquisition front-end processor is configured to acquire the medical image data imported from the PACS database interface and transmit the medical image data to the data sending front-end processor through the one-way isolation gatekeeper, the one-way isolation gatekeeper is configured to ensure that the medical image data is not returned to the PACS database interface, the data sending front-end processor is configured to store the acquired medical image data into the database via the administrator control module, and the firewall security device is configured to ensure security of the database.

The teaching video module may include a teaching video establishment module and a teaching video classification module. The teaching video establishment module and the teaching video classification module are connected to the management side control module, the teaching video establishment module is configured to generate the teaching video according to the medical image data, and the teaching video classification module is configured to classify the teaching video according to types of the teaching video.

The teaching video application module may include a pulmonary digital medical image module, an oral digital medical image module, a cardiac digital medical image module, a brain digital medical image module, an abdominal digital medical image module, an orthopedic digital medical image module, a gynecological digital medic al image module, a 3D printing modeling module, and a VR surgical image training module. The pulmonary digital medical image module is configured to display a pulmonary medical image teaching video, the oral digital medical image module is configured to display an oral medical image teaching video, the cardiac digital medical image module is configured to display a cardiac digital medical image teaching video, the brain digital medical image module is configured to display a brain digital medical image teaching video, the abdominal digital medical image module is configured to display an abdominal medical image teaching video, the orthopedic digital medical image module is configured to display an orthopedic digital medical image teaching video, the gynecological digital medical image module is configured to display a gynecological digital medical image teaching video, the 3D printing modeling module is configured to implement 3D printing modeling, and the VR surgical image training module is configured to display a VR surgical image training teaching video.

The digital medical image teaching and research system further may include a scientific research side. The scientific research side may include a scientific research personnel login module, a scientific research control module, a scientific research log module, and a scientific research module. The scientific research personnel login module, the scientific research log module, the scientific research module, and the central processing unit are connected to the scientific research control module, the scientific research personnel login module is for scientific research personnel to register and log in, the scientific research module is for the scientific research personnel to acquire and perform scientific research on the medical image data, and the scientific research log module is configured to view a scientific research operation record on the medical image data.

The specific process is as follows: the scientific research control module sends an instruction to the central processing unit, the central processing unit sends the instruction to the management side control module, the management side control module acquires the corresponding medical image data and sends same to the management side control module through the central processing unit, and the management side control module sends the medical image data to the scientific research module and controls the scientific research log module to perform operation recording.

The learning side, the management side, and the scientific research side may all be implemented through a personal computer (PC) terminal.

The digital medical image teaching and research system provided by the present disclosure may include a management side, a learning side, a scientific research side, and a central processing unit. The management side is configured to acquire medical image data and generate a teaching video for scientific research personnel to perform scientific research and learning personnel to learn. The management side may include an administrator login module, a management side control module, a teaching video module, a database, and a network connection module. The administrator login module is for an administrator to manage account registration and login. The network connection module is configured to acquire the medical image data in a hospital PACS database. The database is configured to store the medical image data. The teaching video module is configured to generate the teaching video. The learning side is used for learning of the teaching video. The learning side may include a learning login module, a teaching video application module, and a learning side control module. The learning login module is configured to register and login a learning account. The teaching video application module is configured to acquire and play the teaching video. The scientific research side is for the scientific research personnel to perform scientific research. The scientific research side may include a scientific research personnel login module, a scientific research control module, a scientific research log module, and a scientific research module. The scientific research personnel login module is for the scientific research personnel to register and log in. The scientific research module is configured to acquire and perform scientific research on the medical image data. The scientific research log module is configured to view a scientific research operation record on the medical image data. The system may implement teaching and research based on medical images, may be easy to use, and may improve the efficiency of learning and scientific research.

Specific examples are used herein to explain the principles and implementations of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of the present specification shall not be construed as limitations to the present disclosure.

Various embodiments of the disclosure may have one or more of the following effects. In some embodiments, the disclosure provides a digital medical image teaching and research system which integrates scientific research and teaching and provides reference for teaching and research through the medical image data in a hospital picture archiving and communication system (PACS) database. The system may be easy to use and may improve the learning efficiency of medical personnel. In other embodiments, the disclosure may provide a digital medical image teaching and research system which integrates scientific research and teaching, which may provide reference for teaching and research through medical image data in a hospital picture archiving and communication system (PACS) database, which may be convenient to use, and which may improve the learning efficiency of medical personnel.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A digital medical image teaching and research system, comprising a management side, a learning side, and a central processing unit, wherein:
both the management side and the learning side are connected to the central processing unit;
the management side comprises an administrator login module, a management side control module, a teaching video module, a database, and a network connection module, wherein:
the administrator login module, the teaching video module, the database, and the central processing unit are connected to the management side control module;
the management side control module is connected to a hospital picture archiving and communication system (PACS) database through the network connection module;
the administrator login module is configured to manage account registration and login by an administrator;
the network connection module is configured to acquire medical image data in the hospital PACS database;
the database is configured to store the medical image data acquired by the network connection module; and
the teaching video module is configured to generate a teaching video according to the medical image data; and
the learning side comprises a learning login module, a teaching video application module, and a learning side control module, wherein:
the learning login module, the teaching video application module, and the central processing unit are connected to the learning side control module;
the learning login module is configured for a learning personnel to register and login a learning account; and
the teaching video application module is configured to acquire and to play the teaching video.

2. The digital medical image teaching and research system according to claim 1, wherein:
the network connection module comprises a data acquisition front-end processor, a one-way isolation gatekeeper, a data sending front-end processor, and a firewall security device;
the hospital PACS database is connected to the data acquisition front-end processor through a PACS database interface;
the data acquisition front-end processor is connected to the one-way isolation gatekeeper;
the one-way isolation gatekeeper is connected to the data sending front-end processor;
the data sending front-end processor is connected to the firewall security device;
the firewall security device is connected to an administrator control module;
the data acquisition front-end processor is configured to acquire the medical image data imported from the PACS database interface and to transmit the medical image data to the data sending front-end processor through the one-way isolation gatekeeper;
the one-way isolation gatekeeper is configured to ensure that the medical image data is not returned to the PACS database interface;
the data sending front-end processor is configured to store the acquired medical image data into the database via the administrator control module; and
the firewall security device is configured to ensure security of the database.

3. The digital medical image teaching and research system according to claim 2, wherein:
the teaching video module comprises a teaching video establishment module and a teaching video classification module;
the teaching video establishment module and the teaching video classification module are connected to the management side control module;
the teaching video establishment module is configured to generate the teaching video according to the medical image data; and
the teaching video classification module is configured to classify the teaching video according to types of the teaching video.

4. The digital medical image teaching and research system according to claim 1, wherein:
the teaching video application module comprises a pulmonary digital medical image module, an oral digital medical image module, a cardiac digital medical image module, a brain digital medical image module, an abdominal digital medical image module, an orthopedic digital medical image module, a gynecological digital medical image module, a three-dimensional (3D) printing modeling module, and a virtual-reality (VR) surgical image training module;
the pulmonary digital medical image module is configured to display a pulmonary medical image teaching video;
the oral digital medical image module is configured to display an oral medical image teaching video;
the cardiac digital medical image module is configured to display a cardiac digital medical image teaching video;
the brain digital medical image module is configured to display a brain digital medical image teaching video;
the abdominal digital medical image module is configured to display an abdominal medical image teaching video;
the orthopedic digital medical image module is configured to display an orthopedic digital medical image teaching video;
the gynecological digital medical image module is configured to display a gynecological digital medical image teaching video;
the 3D printing modeling module is configured to implement 3D printing modeling; and
the VR surgical image training module is configured to display a VR surgical image training teaching video.

5. The digital medical image teaching and research system according to claim 1, further comprising a scientific research side, wherein:
the scientific research side comprises a scientific research personnel login module, a scientific research control module, a scientific research log module, and a scientific research module;
the scientific research personnel login module, the scientific research log module, the scientific research module, and the central processing unit are connected to the scientific research control module;
the scientific research personnel login module is configured for a scientific research personnel to register and log in;
the scientific research module is configured for the scientific research personnel to acquire and perform scientific research on the medical image data; and
the scientific research log module is configured to view a scientific research operation record on the medical image data.
